SEQUENCE LISTING (10 sequences)

SEQ ID NO: 1 - DNA, 1563 bp, Zea mays
atggccatcatactcgtacgagcagcgtcgccggggctctccgccgccgacagcatcagccaccaggggactctccagtgctccaccctgctcaagacgaagaggccggcggcgcggcggtggatgccctgctcgctccttggcctccacccgtgggaggctggccgtccctcccccgccgtctactccagcctgcccgtcaacccggcgggagaggccgtcgtctcgtccgagcagaaggtctacgacgtcgtgctcaagcaggccgcattgctcaaacgccagctgcgcacgccggtcctcgacgccaggccccaggacatggacatgccacgcaacgggctcaaggaagcctacgaccgctgcggcgagatctgtgaggagtatgccaagacgttttacctcggaactatgttgatgacagaggagcggcgccgcgccatatgggccatctatgtgtggtgtaggaggacagatgagcttgtagatgggccaaacgccaactacattacaccaacagctttggaccggtgggagaagagacttgaggatctgttcacgggacgtccttacgacatgcttgatgccgctctctctgataccatctcaaggttccccatagacattcagccattcagggacatgattgaagggatgaggagtgatcttaggaagacaaggtataacaacttcgacgagctctacatgtactgctactatgttgctggaactgtcgggttaatgagcgtacctgtgatgggcatcgcaaccgagtctaaagcaacaactgaaagcgtatacagtgctgccttggctctgggaattgcgaaccaactcacgaacatactccgggatgttggagaggatgctagaagaggaaggatatatttaccacaagatgagcttgcacaggcagggctctctgatgaggacatcttcaaaggggtcgtcacgaaccggtggagaaacttcatgaagaggcagatcaagagggccaggatgttttttgaggaggcagagagaggggtaaatgagctctcacaggctagcagatggccagtatgggcttccctgttgttgtacaggcagatcctggatgagatcgaagccaacgactacaacaacttcacgaagagggcgtatgttggtaaagggaagaagttgctagcacttcctgtggcatatggaaaatcgctactgctcccatgttcattgagaaatggccagacctagccaccagagaagctgcaatgcaaggttcaggttaggctagatagaaagttaaatggggcaacatcaggaggccttgatgaaaaacagacaacctggtgaattgttgttgggatcaggcacagaacagataagagccgcgcagccaacctagggcatgtttggtttcaattagttctaggactaaactttagtcctaggactaaactttagtccctatatgtttggttctagggactaaatagattctaaagtcattaaatacattgtccaaagactcaaatacccttagaatatactcatgatattagttatctataaaaaaaaaaaaaa

SEQ ID NO: 2 - DNA, 56 bp, Zea mays
ggactaaatagattctaaagtcattaaatacattgtccaaagactcaaataccctt

SEQ ID NO: 3 - DNA, 74 bp, Artificial Sequence
  Forward primer sequence for phytosene synthase
ttaaatacattgaccaaagactcaatttttttaaattgagtctttggtcaatgtatttaagcgcgttttcgcgc

SEQ ID NO: 4 - DNA, 20 bp, Artificial Sequence
  Primer sequence for phytosene synthase
gttaaatggggcaacatcag

SEQ ID NO: 5 - DNA, 18 bp, Artificial Sequence
  Reverse primer sequence for phytosene synthase
cagagtatattctaaggg

SEQ ID NO: 6 - DNA, 1462 bp, Zea mays
  misc_feature 1409: n = A,T,C or G
atggcttctgaggttgtccgccctagcgacgccgagctactaaaggcacaagccgacatatggcggctcagcctctcctacctcacgcccctgtcactgaggtgcgccgtggagcttggcatccctacggcgatctaccgccatggcggggctgcctcagccgctgagctggtcacggcgctgtctctcccctccactaagctgccgttcctccgccgcctgctgcggctcctcgccgcgtcgggcgtgttcaccgtcgacaagcagagctcggaggaggagcggtaccgcatcagccccgtgtcctacctcctggtggacggcatccctcacgaggaccacatgaaccacaccgctctggtgctcacctgcacctcgacgcgctacatcgaggcggggatagggctggcggagtggttcaagagggacgtggtcacgtcaccgttcgaggagctgcacggggcgactctgttccacgagagcatggggagcctcgacgcagatttccacgacatggcgagcgaggccttggacgcccacgacaacttcggcatcgagatagctatgcgggagttcagggacttgttcgaggggatccagtccatgacctactgctgcggtaacttcggtgatgacaagggtgccagggccattgtcaaagccttcccacacataaaatgcactgtgctggcccctcccaagatcattgccaccaaaccggccgatggagcgatgataaactatgtcgaaggtgacatgttcagtttcattccacctgctcaaactgtcgtgctaaagcttgtgctgcaccacctgaccgatgaggagtgcgtcaagctcctggctcaatgccggaaggcgattccttcacggaaggatggcgggaaagttataatcggtgatattatgattgaccactctgggcctatgctggaaactcacctcctgatggacatcggcatgatgaccatgacaaaaggacggcagagagatgaaaaagaatggagcgagctcttcaccaaagcagggttcagcgaatacaagatcctgaaggaatttggagctcgtgttgccttcgaagtctatccgtgagattgcacctggactatacatatatatttacatacatatatgtatgtgtgtgggtgccttgcgtggcttggcgtgatgatgtcataaataaagagggtgggtgtgcttgcggtattcatggatggccatggatctgagcacatgacgttggatgctttcttaaggctctcgtggatcttctatatttggaagaagctgtaaaataatatatcgccatctagtgttggcgttggcttgtatgtactttgtacctgctttgttattttgtgtaatatcgagtaacctaatgtatacttgcttactactatcactaccgnaaatctatctctttgccgagtaaaaaaaaaaaaaaaaaaaaaaaaaaaaaaaa

SEQ ID NO: 7 - DNA, 56 bp, Zea mays
ggactatacatatatatttacatacatatatgtatgtgtgtgggtgccttgcgtgg

SEQ ID NO: 8 - DNA, 74 bp, Artificial Sequence
  Oligonucleotide sequence for modification of safener-binding gene
acatatatgtatctgtgtgggtgcctttttttaaaggcacccacacagatacatatatgtgcgcgttttcgcgc

SEQ ID NO: 9 - DNA, 22 bp, Artificial Sequence
  Forward primer for safener-binding gene
ttatgattgaccactctgggcc

SEQ ID NO: 10 - DNA, 24 bp, Artificial Sequence
  Reverse primer for safener-binding gene
ccacgagagccttaagaaagcatc

(19) United States
(12) Patent Application Publication
(10) Pub. No.: US 2004/0205840 A1
(43) Pub. Date: Oct. 14, 2004

(54) Method for reducing gene expression

(75) Inventors: Pramod B. Mahajan, Urbandale, IA (US); Priya Kannan, Ankeny, IA (US)

Correspondence Address: PIONEER HI-BRED INTERNATIONAL INC., 7100 N.W. 62ND AVENUE, P.O. BOX 1000, JOHNSTON, IA, 50131, US

(73) Assignee: Pioneer Hi-Bred International, Inc.

(21) Appl. No.: 10/805,853

(22) Filed: Mar. 22, 2004

Related U.S. Application Data
(60) Provisional application No. 60/462,446, filed on Apr. 11, 2003.

(51) Int. Cl. A01H 1/00; C12N 15/82; C12N 5/04

(52) U.S. Cl. 800/278; 435/468; 435/419

(57) ABSTRACT

The invention provides novel methods and compositions for reducing plant gene expression. Included are plants, plant cells, and seed containing a polynucleotide with reduced expression.

### CROSS-REFERENCE TO RELATED APPLICATIONS

This application claims priority to U.S. Provisional Application Serial No. 60/462,446 filed Apr. 11, 2003, the disclosure of which is incorporated herein by reference.

### FIELD OF INVENTION

The present invention related generally to plant molecular biology.

### BACKGROUND

Insertional mutagenesis, antisense inhibition or inhibition via interference by double-stranded RNA (also known as RNAi technology) are some of the methods for silencing of plant genes. These methods are both labor intensive and very costly. Availability of alternative technologies for reducing plant gene expression is highly desirable for generating transgenic crops with improved agronomic or grain traits. Gene silencing as a tool for regulation of gene expression may be used on endogenous genes, modified endogenous genes or transgenes.

### SUMMARY OF THE INVENTION

### DETAILED DESCRIPTION OF THE INVENTION

Definitions

The term “isolated” refers to material, such as a nucleic acid or a protein, which is: (1) substantially or essentially free from components which normally accompany or interact with the material as found in its naturally occurring environment or (2) if the material is in its natural environment, the material has been altered by deliberate human intervention to a composition and/or placed at a locus in the cell other than the locus native to the material, including alteration of a single base pair.

As used herein, the term “plant” includes reference to whole plants, plant organs (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen, and microspores. The class of plants, which can be used in the methods of the invention, is generally as broad as the class of higher plants amenable to transformation techniques, including both monocotyledonous and dicotyledonous plants. Useful plants include, but are not limited to maize, soybean, sunflower, sorghum, canola, wheat, alfalfa, cotton, rice, barley, and millet.

As used herein “transformation” includes stable transformation and transient transformation unless indicated otherwise.

As used herein “stable transformation” refers to the transfer of a nucleic acid fragment into a genome of a host organism (this includes both nuclear and organelle genomes) resulting in genetically stable inheritance. In addition to traditional methods, stable transformation includes the alteration of gene expression by any means including oligonucleotide directed sequence modification or transposon insertion.

As used herein “transient transformation” refers to the transfer of a nucleic acid fragment or protein into the nucleus (or DNA-containing organelle) of a host organism resulting in gene expression without, necessarily, resulting in integration and stable inheritance.

As used herein “expression cassette” refers to a nucleic acid construct, generated recombinantly or synthetically, with a series of specified nucleic acid elements, which permit transcription of a particular nucleic acid in a host cell. The expression cassette can be incorporated into a plasmid, chromosome, mitochondrial DNA, plastid DNA, virus, or nucleic acid fragment. Typically, the expression cassette portion of an expression vector includes, among other sequences, a nucleic acid to be transcribed, and a promoter.

As used herein “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and where necessary to join two protein coding regions, contiguous and in the same reading frame.

As used herein, “target gene” or “target polynucleotide” refers to any polynucleotide suitable for regulation of expression, including both endogenous chromosomal genes and transgenes, as well as episomal or extrachomosomal genes, mitochondrial genes, chloroplastic genes, viral genes, bacterial genes, animal genes, plant genes, protozoal genes and fungal genes. A target gene or polynucleotide can be found in either the nuclear or organelle genomes. A target gene or polynucleotide includes, but is not limited to, the 5′ untranslated region, the 3′ untranslated region, the coding region, an intron, an exon, secretory regions or any other part of the polynucleotide that is transcribed into RNA.

As used herein “target polynucleotide”, “target sequence”, or “target site” refers to the polynucleotide to be modified in the host organism.

As used herein a “RNA destabilizing sequence element” or “RDS element” refers to a polynucleotide containing sequences ATAGAT and GTA, which are capable of destabilizing RNA. In addition, a third domain may be included that contains the sequence GGA. The three domains can be placed in the following 5′ to 3′ order of: GGA, followed by ATAGAT, followed by GTA. These elements may be found or introduced into any part of a gene including, but not limited to, the 5′ untranslated region, the 3′ untranslated region, the coding region, an intron, an exon, secretory regions or any other part of the nucleotide that is transcribed into RNA. The RDS element could be positioned any where from the start of transcription to within 19 base pairs of the poly A tail. However, the RDS element is generally at least 19 base pairs upstream from the poly A tail of the gene. Alternatively the RDS element could be at least 19 to 50, 19 to 70, 50 to 100, 100 to 250, 250 to 500, 500 to 1000, 1000 to 2000 base pairs or 19 base pairs from the poly A tail to the start of transcription. In addition, the RDS element could be between 1 to 50, 50 to 100, 100 to 200, 200 to 500, 500 to 1000, 1000 to 2000, 2000 to 3000, 3000 to 5000, 5000 to 10,000 base pairs from the start of transcription.

In addition, the distance between the two conserved domains of ATAGAT and GTA can be at least 1, 2, 5, 10, 11, 12, 13, 14, or 15 base pairs. Alternatively, the distance between the two conserved domains of ATAGAT and GTA could be 1 to 50, 1 to 75 or 1 to 100 base pairs. The distance between GGA and the two conserved domains of ATAGAT and GTA could be 1, 2, 3, 4, 5, 6, 7, 8, 9, 10, 11, 12, 13, 14, or 15 base pairs. Alternatively, the distance between the GGA and the two conserved domains of ATAGAT and GTA could be 1 to 50, 1 to 75 or 1 to 100 base pairs.

The number of RDA elements in a gene may vary. Although a gene must contain at least one RDA element to cause destabilization of the mRNA, a gene may contain, two or more copies of the RDA element, such as two, three, four, five or more copies of the RDA element. In addition, a gene containing at least one RDA element could be modified to contain one or more additional RDA elements. The introduction of additional RDA elements, within a gene, may further enhance the destabilization of the mRNA of the gene.

As used herein, “targeted gene modification” refers to any process whereby a specific sequence modification is facilitated at a desired genetic locus by a transforming nucleic acid.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells whether or not its origin is a plant cell. Exemplary plant promoters include, but are not limited to, those that are obtained from plants, plant viruses, and bacteria which comprise genes expressed in plant cells such as Agrobacterium or Rhizobium. Examples of promoters under developmental control include promoters that preferentially initiate transcription in certain tissues, such as leaves, roots, or seeds. Such promoters are referred to as “tissue preferred”. Promoters who initiate transcription only in certain tissue are referred to as “tissue specific”. A “cell type” specific promoter primarily drives expression in certain cell types in one or more organs, for example, vascular cells in roots or leaves. An “inducible” or “repressible” promoter is a promoter, which is under environmental control. Examples of environmental conditions that may effect transcription by inducible promoters include anaerobic conditions or the presence of light. Tissue specific, tissue preferred, cell type specific, and inducible promoters constitute the class of “non-constitutive” promoters. A “constitutive” promoter is a promoter, which is active under most environmental conditions.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The term “selectively hybridizes” includes a reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, 90% sequence identity, and 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20× SSC=3.0 M NaC1/0.3 M trisodium citrate) at 50 to 55° C. Exemplary moderate stringency conditions include hybridization in 40 to 45% formamide, 1 M NaC1, 1% SDS at 37° C., and a wash in 0.5× to 1×SSC at 55 to 60° C. Exemplary high stringency conditions include hybridization in 50% formamide, 1 M NaC1, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6 (log M)+0.41 (% CG)−0.61 (% form)−500/L; where M is the molarity of monovalent cations, % CG is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) then the SSC concentration can be increased so that a higher temperature can be used. An extensive guide to the hybridization of nucleic acids is found in Tijssen, Laboratory Techniques in Biochemistry and Molecular Biology-Hybridization with Nucleic Acid Probes, Part I, Chapter 2 “Overview of principles of hybridization and the strategy of nucleic acid probe assays”, Elsevier, N.Y. (1993); and Current Protocols in Molecular Biology, Chapter 2, Ausubel et al., Eds., Greene Publishing and Wiley-Interscience, New York (1995).

As used herein, “transgenic plant” includes reference to a plant, which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, spontaneous mutation, or oligonucleotide induced genomic modification.

As used herein, “vector” includes reference to a nucleic acid used in transfection of a host cell and into which can be inserted a polynucleotide. Vectors are often replicons. Expression vectors permit transcription of a nucleic acid inserted therein.

Methods of alignment of sequences for comparison are well known in the art. Optimal alignment of sequences for comparison may be conducted by the local homology algorithm of Smith and Waterman. Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG) (GCG® programs, Accelrys, Inc., San Diego, Calif.); the CLUSTAL program is well described by Higgins and Sharp, Gene 73:237-244 (1988); Higgins and Sharp, CABIOS 5:151-153 (1989); Corpet et al., Nucleic Acids Research 16:10881-90 (1988); Huang et al., Computer Applications in the Biosciences 8:155-65 (1992), and Pearson et al., Methods in Molecular Biology 24:307-331 (1994), Current Protocols in Molecular Biology, Chapter 19, Ausubel et al., Eds., Greene Publishing and Wiley-Interscience, New York (1995).

GAP uses the algorithm of Needleman and Wunsch (J. Mol. Biol. 48:443453 (1970)) to find the alignment of two complete sequences that maximizes the number of matches and minimizes the number of gaps. GAP considers all possible alignments and gap positions and creates the alignment with the largest number of matched bases and the fewest gaps. Default gap creation penalty values and gap extension penalty values in Version 10 of the Wisconsin Genetics Software Package are 8 and 2, respectively, for protein sequences. For nucleotide sequences the default gap creation penalty is 50 while the default gap extension penalty is 3. The gap creation and gap extension penalties can be expressed as an integer selected form the group of integers consisting of form 0 to 100. Thus, for example, the gap creation and gap extension penalties can be 0, 1, 2, 3, 4, 5, 6, 7, 8, 9, 10, 15, 20, 30, 40, 50, 60, or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff and Henikoff, Proc. Natl. Acad. Sci. USA 89:10915). Unless otherwise stated, sequence identity/similarity values provided herein refer to the value obtained using the GAP version 10 of Wisconsin Genetic Software Package using default parameters.

In general, the invention provides for compositions and methods for decreasing gene expression of a targeted gene in a cell. The method involves converting or modifying a nucleotide sequence of interest in a cell, by targeted gene modification, to contain a RDS element. The modified nucleotide sequence then produces an unstable mRNA, which is targeted for rapid decay. It is generally accepted that stability of a specific messenger RNA largely determines the steady state levels of that mRNA, and directly affects expression of the cognate protein. Modulating steady state levels of mRNA has been used as an effective means of controlling over-expression of proteins in plants. Therefore, reduction in the levels of a specific mRNA would lead to reduction in expression or silencing of a specific gene.

Expression of a transgene in plants can be altered by the presence of a specific sequence normally present in the 3′ untranslated region of that gene. This sequence is called the downstream element or DST (see Newman et. al., Plant Cell 5:701-714 (1993)). The DST element is approximately 45 nucleotides in length and contains three highly conserved motifs separated by two variable regions. Mutagenesis studies have shown that, residues within two conserved domains, ATAGAT and GTA, are capable of making a specific mRNA molecule unstable, leading to reduction in the level of expression of the cognate gene. A third domain, GGA, positioned 5′ to the two conserved domains, may also be involved in the DST element.

Modifying a portion of a transgene or exogenous gene to contain either two conserved domains ATAGAT and GTA or three conserved domains GGA, ATAGAT and GTA, will create an RDS element. By creating at least one RDS element within a gene, the mRNA would become unstable and thereby reduce or silence the expression of the gene. Alternatively, multiple RDS elements may be introduced into a gene. The RDS element or elements could be created anywhere within the gene that is transcribed into RNA.

Selecting a Gene for Modification

Any gene can be modified to contain an active RDS element, including but not limited to, genes encoding agronomic traits, insect resistance, disease resistance, herbicide resistance, sterility, grain characteristics, and commercial products. The gene can be an endogenous gene, a modified endogenous gene or a transgene. Genes of interest also include those involved in oil, starch, carbohydrate, or nutrient metabolism as well as those affecting, for example, kernel size, sucrose loading, and the like. The quality of grain is reflected in traits such as levels and types of oils, saturated and unsaturated, quality and quantity of essential amino acids, and levels of cellulose.

Sterility genes can also be targeted, including male tissue-preferred genes and genes with male sterility phenotypes such as QM, described in U.S. Pat. No. 5,583,210. Other genes include kinases and those encoding compounds toxic to either male or female gametophytes.

Possible target genes include but are not limited to:

(A) Plant disease resistance genes. Plant defenses are often activated by specific interaction between the product of a disease resistance gene (R) in the plant and the product of a corresponding avirulence (Avr) gene in the pathogen. A plant variety can be transformed with a cloned resistance gene to engineer plants that are resistant to specific pathogen strains. See, for example Jones et al., Science 266:789 (1994) (cloning of the tomato Cf-9 gene for resistance to Cladosporium fulvum); Martin et al., Science 262:1432 (1993) (tomato Pto gene for resistance to Pseudomonas syringae pv. tomato encodes a protein kinase); Mindrinos et al., Cell 78:1089 (1994) (Arabidopsis RSP2 gene for resistance to Pseudomonas syringae).

(B) A gene conferring resistance to a pest, such as soybean cyst nematode. See e.g. PCT Application WO96/30517; PCT Application WO93/19181.

(C) A Bacillus thuringiensis protein, a derivative thereof or a synthetic polypeptide modeled thereon. See, for example, Geiser et al., Gene 48: 109 (1986), who disclose the cloning and nucleotide sequence of a Bt δ-endotoxin gene. Moreover, DNA molecules encoding δ-endotoxin genes can be purchased from American Type Culture Collection (Manassas, Va.), for example, under ATCC Accession Nos. 40098, 67136, 31995 and 31998.

(D) A lectin. See, for example, the disclosure by Van Damme et al., Plant Molec. Biol. 24:25 (1994), who disclose the nucleotide sequences of several Clivia miniata mannose-binding lectin genes.

(E) A vitamin-binding protein such as avidin. See PCT Application US93/06487, the contents of which are hereby incorporated by reference. The application teaches the use of avidin and avidin homologues as larvicides against insect pests.

(F) An enzyme inhibitor, for example, a protease or proteinase inhibitor or an amylase inhibitor. See, for example, Abe et al., J. Biol. Chem. 262:16793 (1987) (nucleotide sequence of rice cysteine proteinase inhibitor), Huub et al., Plant Molec. Biol. 21:985 (1993) (nucleotide sequence of cDNA encoding tobacco proteinase inhibitor 1), Sumitani et al., Biosci. Biotech. Biochem. 57:1243 (1993) (nucleotide sequence of Streptomyces nitrosporeus alpha-amylase inhibitor) and U.S. Pat. No. 5,494,813 (Hepher and Atkinson, issued Feb. 27, 1996).

(G) An insect-specific hormone or pheromone such as an ecdysteroid and juvenile hormone, a variant thereof, a mimetic based thereon, or an antagonist or agonist thereof. See, for example, the disclosure by Hammock et al., Nature 344:458 (1990), of baculovirus expression of cloned juvenile hormone esterase, an inactivator of juvenile hormone.

(H) An insect-specific peptide or neuropeptide which, upon expression, disrupts the physiology of the affected pest. For example, see the disclosures of Regan, J. Biol. Chem. 269:9 (1994) (expression cloning yields DNA coding for insect diuretic hormone receptor), and Pratt et al., Biochem. Biophys. Res. Comm. 163:1243 (1989) (an allostatin is identified in Diploptera puntata). See also U.S. Pat. No. 5,266,317 to Tomalski et al., who disclose genes encoding insect-specific, paralytic neurotoxins.

(I) An insect-specific venom produced in nature by a snake, a wasp, etc. For example, see Pang et al., Gene 116:165 (1992), for disclosure of heterologous expression in plants of a gene coding for a scorpion insectotoxic peptide.

(J) An enzyme responsible for an hyperaccumulation of a monterpene, a sesquiterpene, a steroid, hydroxamic acid, a phenylpropanoid derivative or another non-protein molecule with insecticidal activity.

(K) An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT Application WO 93/02197 in the name of Scott et al., which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23:691 (1993), who teach the nucleotide sequence of a cDNA encoding tobacco hookworm chitinase, and Kawalleck et al., Plant Molec. Biol. 21:673 (1993), who provide the nucleotide sequence of the parsley ubi4-2 polyubiquitin gene.

(L) A molecule that stimulates signal transduction. For example, see the disclosure by Botella et al., Plant Molec. Biol. 24:757 (1994), of nucleotide sequences for mung bean calmodulin cDNA clones, and Griess et al., Plant Physiol. 104:1467 (1994), who provide the nucleotide sequence of a maize calmodulin cDNA clone.

(M) A hydrophobic moment peptide. See PCT Application WO95/16776 (disclosure of peptide derivatives of Tachyplesin which inhibit fungal plant pathogens) and PCT application WO95/18855 (teaches synthetic antimicrobial peptides that confer disease resistance), the respective contents of which are hereby incorporated by reference.

(N) A membrane permease, a channel former or a channel blocker. For example, see the disclosure by Jaynes et al., Plant Sci. 89:43 (1993), of heterologous expression of a cecropin-β lytic peptide analog to render transgenic tobacco plants resistant to Pseudomonas solanacearum.

(O) A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. Rev. Phytopathol. 28:451 (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

(P) An insect-specific antibody or an immunotoxin derived therefrom. Thus, an antibody targeted to a critical metabolic function in the insect gut would inactivate an affected enzyme, killing the insect. Cf. Taylor et al., Abstract #497, Seventh Int'l Symposium on Molecular Plant-Microbe Interactions (Edinburgh, Scotland, 1994) (enzymatic inactivation in transgenic tobacco via production of single-chain antibody fragments).

(Q) A virus-specific antibody. See, for example, Tavladoraki et al., Nature 366:469 (1993), who show that transgenic plants expressing recombinant antibody genes are protected from virus attack.

(R) A developmental-arrestive protein produced in nature by a pathogen or a parasite. Thus, fungal endo α-1,4-D-polygalacturonases facilitate fungal colonization and plant nutrient release by solubilizing plant cell wall homo-α-1,4-D-galacturonase. See Lamb et al., Bio/Technology 10:1436 (1992). The cloning and characterization of a gene which encodes a bean endopolygalacturonase-inhibiting protein is described by Toubart et al., Plant J. 2:367 (1992).

(S) A developmental-arrestive protein produced in nature by a plant. For example, Logemann et al., Bio/Technology 10:305 (1992), have shown that transgenic plants expressing the barley ribosome-inactivating gene have an increased resistance to fungal disease.

2. Genes That Confer Resistance To A Herbicide, For Example:

(A) A herbicide that inhibits the growing point or meristem, such as an imidazalinone or a sulfonylurea. Exemplary genes in this category code for mutant ALS and AHAS enzyme as described, for example, by Lee et al., EMBO J. 7:1241 (1988), and Miki et al., Theor. Appl. Genet. 80:449 (1990), respectively.

(B) Glyphosate (resistance imparted by mutant 5-enolpyruvl-3-phosphikimate synthase (EPSP), aroA, and GAT genes) and other phosphono compounds such as glufosinate (phosphinothricin acetyl transferase, PAT) and Streptomyces hygroscopicus phosphinothricin-acetyl transferase, bar, genes), and pyridinoxy or phenoxy propionic acids and cycloshexones (ACCase inhibitor-encoding genes). See, for example, U.S. Pat. No. 4,940,835 to Shah et al., which discloses the nucleotide sequence of a form of EPSPS, which can confer glyphosate resistance. Also see U.S. Pat. No. 5,627,061 to Barry et al. which describes genes encoding EPSPS enzymes. A DNA molecule encoding a mutant aroA gene can be obtained under ATCC Accession No. 39256, and the nucleotide sequence of the mutant gene is disclosed in U.S. Pat. No. 4,769,061 to Comai. See also, PCT Application WO 02/36782 describing the GAT gene and its use for herbicide resistance. European Patent Application No. 0 333 033 to Kumada et al. and U.S. Pat. No. 4,975,374 to Goodman et al. disclose nucleotide sequences of glutamine synthetase genes which confer resistance to herbicides such as L-phosphinothricin. The nucleotide sequence of a phosphinothricin-acetyl-transferase gene is provided in European Application No. 0 242 246 to Leemans et al. De Greef et al., Bio/Technology 7:61 (1989), describe the production of transgenic plants that express chimeric bar genes coding for phosphinothricin acetyl transferase activity. Exemplary of genes conferring resistance to phenoxy propionic acids and cycloshexones, such as sethoxydim and haloxyfop, are the Acc1-S1, Acc1-S2 and Acc1-S3 genes described by Marshall et al., Theor. Appl. Genet. 83:435 (1992).

(C) A herbicide that inhibits photosynthesis, such as a triazine (psbA and gs+genes) and a benzonitrile (nitrilase gene). Przibilla et al., Plant Cell 3:169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441 and 67442. Cloning and expression of DNA coding for a glutathione S-transferase is described by Hayes et al., Biochem. J. 285:173 (1992).

3. Genes That Confer Or Contribute To A Value-Added Trait, Such As:

(A) Modified fatty acid metabolism, for example, by transforming a plant with an antisense gene of stearoyl-ACP desaturase to increase stearic acid content of the plant. See Knultzon et al., Proc. Natl. Acad. Sci. USA 89:2624 (1992).

(B) Decreased phytate content

(1) Introduction of a phytase-encoding gene would enhance breakdown of phytate, adding more free phosphate to the transformed plant. For example, see Van Hartingsveldt et al., Gene 127:87 (1993), for a disclosure of the nucleotide sequence of an Aspergillus niger phytase gene.

(2) A gene could be introduced that reduces phytate content. In maize, this, for example, could be accomplished, by cloning and then reintroducing DNA associated with the single allele which is responsible for maize mutants characterized by low levels of phytic acid. See Raboy et al., Maydica 35:383 (1990).

(C) Modified carbohydrate composition effected, for example, by transforming plants with a gene coding for an enzyme that alters the branching pattern of starch. See Shiroza et al., J. Bacteriol. 170: 810 (1988) (nucleotide sequence of Streptococcus mutans fructosyltransferase gene), Steinmetz et al., Mol. Gen. Genet. 200:220 (1985) (nucleotide sequence of Bacillus subtilis levansucrase gene), Pen et al., Bio/Technology 10:292 (1992) (production of transgenic plants that express Bacillus licheniformis alpha-amylase), Elliot et al., Plant Molec. Biol. 21:515 (1993) (nucleotide sequences of tomato invertase genes), Søgaard et al., J. Biol. Chem. 268:22480 (1993) (site-directed mutagenesis of barley alpha-amylase gene), and Fisher et al., Plant Physiol. 102:1045 (1993) (maize endosperm starch branching enzyme II).

While any gene can be modified to decrease expression, genes having a partial match to the RDS element will be easiest to modify. A gene having only a one or two base pair mismatch to the RDS element will be the simplest to modify. To identify genes having only a few base pair mismatch any conventional sequence analysis method can be used, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5: 151-153 (1989); Corpet et al., Nucleic Acids Research 16:10881-90 (1988); Huang et al., Computer Applications in the Biosciences 8:155-65 (1992), and Pearson et al., Methods in Molecular Biology 24:307-331 (1994). In addition any program that involves direct pattern recognition may be used, such as, but not limited to PSI-Blast (see, Alschul et. al., Nuc. Acid Res. 24:3389-3402 (1997); pattern-hit initiated BLAST (PHI-BLAST) (see, Zhang et. al., Nuc. Acid Res. 26:3986-3990 (1998)); and GCG/FindPattern. Alternatively, a custom program, using a computer language such as Perl, could be developed for searching string-related queries. Please see, Torkington and Christiansen, Perl Cookbook: Tips and Tricks for Perl Programmers, O'Reilly, 1998.

For example, using the Psi-Blast format, a consensus sequence was generated from the aligned DST sequences. The consensus sequence contains, from 5′ to 3′, the GGA conserved region followed an elastic region that can be any 4 to 11 nucleotides, followed by the ATAGATT conserved region, followed by another elastic region that can be any 4 to 9 nucleotides, followed by a block that requires either an A or C, then either a T or A, then either a T or A, followed by any two nucleotides, followed by the conserved region GTA, followed by a block that requires either a T or C. Any exceptions in the conserved region are treated as mismatches. This consensus sequence has fourteen specific nucleotide positions plus four nucleotide positions that could be any of two nucleotides. The consensus sequence can be compared to other sequences for perfect matches using any program able to do direct pattern recognition.

Methods of Targeted Gene Modification

Any method capable of targeting and modifying specific nucleotide sequences may be used to produce an RDS element within a particular polynucleotide. Examples of methods that may be used to produce RDS elements within a particular polynucleotide are below.

Site-specific recombination systems maybe used to produce RDS elements and are reviewed in Sauer (1994) Current Opinion in Biotechnology 5:521-527, Nunes-Duby et al. (1998) Nucl. Acids Res. 26:391-406, and Sadowski (1993) FASEB 7:760-767, the contents of which are herein incorporated by reference. Any type of site-specific recombination can be used in the invention. Examples of site-specific recombination systems suitable for this invention include, but are not limited to, the integrase family, such as the FLP/FRT system from yeast, and the Cre/Lox system from bacteriophage P1, as well as the Int, and R systems. The resolvase family can also be used, for example yδ resolvase, and the like. Examples of site-specific recombination systems used in plants can be found in U.S. Pat. No. 5,929,301; U.S. Pat. No. 6,175,056; WO 99/25821; U.S. Pat. No. 6,331,661; U.S. Pat. No. 6,300,545; U.S. Pat. No. 6,262,341; and co-pending U.S. application Ser. No. 10/138,546, the contents of each are herein incorporated by reference.

An alternative method for targeted gene modification is using an oligonucleotide directed sequence modification method. In general, methods utilizing oligonucleotides, use DNA, RNA, or a combination of DNA and RNA. In addition, RNA or DNA analogs may be used in the oligonucleotide. The oligonucleotides can be constructed as single nucleic acid polymers containing at least one region or two regions having at least one base, or two to five bases that are not Watson-Crick paired. The mismatched base or bases is then modified in the plant cell by recombination or DNA repair. One such method is described in WO 99/25853, Zhu et. al., Nature Biotechnology 18:555-558 (2000), and Zhu et al., Proc. Natl. Acad. Sci. USA 96:8768-8773 (1999) (method in plants) and U.S. Pat. No. 5,565,350 and Yoon et al., Proc. Natl. Acad. Sci. USA 93:2071-2076 (1996) (method in mammalian cells), the contents of each are herein incorporated by reference.

Different modified nucleic acids can be used in the present invention. For example, phosphorothioate DNA analogs have been used as oligonucleotides for targeted gene modification. Please see, Liu et al., Nucleic Acids Res. 29:42384250 (2001), which is hereby incorporated by reference. Normally, oligonucleotides containing phosphorothioate DNA analogs are single stranded with the phosphorothioate linkages located at both the 3′ and 5′ termini.

Methods of targeted gene modification, using DNA analogs, also include the use of oligonucleotides that contain cationic phosphoramidite internucleoside linkages (cationic oligonucleotides), as can be found in co-pending U.S. Application No. 60/361,798; the disclosure of which is herein incorporated by reference. The template oligonucleotides are typically single-stranded DNA. Generally the template oligonucleotide will be less than about 200 nucleotides, typically about 20 to about 80 nucleotides, or about 20 to about 60 nucleotides in size. In selecting and preparing the template oligonucleotide, the complementary regions can be complementary to either a transcribed or a non-transcribed strand of the plant genomic DNA.

A portion of the template oligonucleotide will comprise cationic phosphoramidite internucleoside linkages. Methods for preparing cationic oligonucleotides having such linkages are found in Dagle & Weeks, Nucl. Acids Res. 24:2143-2149, 1996; Dagle et al., Nucl. Acids Res 28:2153-2157, 2000; U.S. Pat. No. 6,274,313 and U.S. Pat. No. 5,734,040 the disclosures of which are incorporated herein by reference. The cationic phosphoramidite is incorporated during oligonucleotide synthesis via oxidative amidation. In one embodiment the cationic phosphoramidite have both a primary and a tertiary amine. Positively charged internucleoside linkages can be generated by using a diethylethylenediamine such as N,N-diethylethylenediamine (DEED) and methoxyehtylamine phosphoramidites.

Typically the cationic oligonucleotide will comprise the ends of the template oligonucleotide, however a large portion or even the entire length of the template oligonucleotide can comprise cationic oligonucleotides. Generally the cationic oligonucleotide portion will comprise at least about 10%, 20%, 30%, 40%, 50%, 60%, 70%, 80%, 90% of the template oligonucleotide and up to the entire length of the template oligonucleotide.

It is further recognized that where more than one point mutation is being inserted into the genome, that the template oligonucleotide can be constructed to have more than two flanking complementary sequences. That is, complementary sequences may flank more than one modifying DNA sequence (non-complementary region). Such a design may provide better stability and recombination efficiency where the sites of mutation or modification are not contiguous.

Alternatively, the oligonucleotides could contain a high-affinity DNA analog such as a locked nucleic acid (LNA). LNA/DNA copolymers are more stable and do not degrade as easily in cell extracts as DNA copolymers. The use of LNA oligonucleotides for antisense experiments is described in Wahlestedt et. al., Proc. Natl. Acad. Sci. USA 97:5633-5638 (2000), which is herein incorporated by reference. LNA oligonucleotides could contain all LNA polymers or a mixture of LNA and DNA polymers. In LNA/DNA copolymers, the LNA polymers could be mixed with in the copolymer or arranged with the LNA polymers at the ends of the oligonucleotide to form a LNA/DNA/LNA gap-mer. LNA-containing polymers can be synthesized using an automated DNA synthesizer as described in Singh et. al., Chem. Commun. 1998:455456 (1998) and Koshin et al., Tetrahedron 54:3607-3630 (1998), both of which are hereby incorporated by reference.

Methods for construction of the oligonucleotides of the invention are known in the art. The oligonucleotides can be synthesized by solid phase synthesis. See, Caruthers, M. H. (1985) Science 230:281-285; Itakura et al. (1984) Ann. Rev. Biochem. 53:523-556. Such methods may be modified to permit the synthesis of RNA, RNA-DNA, or modified molecules. See, for example, Scaringe et al. (1990) Nucleic Acids Research 18:5433-5441; Usman et al. (1992) Nucl. Acids Res. 20:6695-6699; Swiderski et al. (1994) Anal. Biochem. 216:83-88; Usman and Cedergren (1992) Trends Biochem. Sci. 17:334-339; Yoon et al. (1996) Proc. Natl. Acad. Sci. USA 93:2071-2076; Cole-Strauss et al. (1996) Science 273:1386-1389; and Kren et al. (1997) Hepatology 25:1462-1468. Such disclosures are herein incorporated by reference. General methods for DNA manipulation are known in the art. See, for example, Molecular Cloning, Sambrook et al. (eds.) Cold Spring Harbor Laboratory Press (1989).

The vectors, template oligonucleotides or constructed oligonucleotides can be introduced into the plant cell by any method available in the art. In this manner, genetically modified plants, plant cells, plant tissue, seed, and the like can be obtained. The template or oligonucleotides may be introduced into the plant by one or more techniques typically used for direct DNA delivery into cells. Such protocols may vary depending on the type of plant or plant cell, i.e. monocot or dicot, targeted for gene modification. Suitable methods of transforming plant cells include microinjection (Crossway et al. (1986) Biotechniques 4:320-334), electroporation (Riggs et al. (1986) Proc. Natl. Acad. Sci. USA 83:5602-5606, direct gene transfer (Paszkowski et al. (1984) EMBO J. 3:2717-2722), and ballistic particle acceleration (see, for example, Sanford et al., U.S. Pat. No. 4,945,050; WO 91/10725 and McCabe et al. (1988) Biotechnology 6:923-926). Also see, Weissinger et al. (1988) Annual Rev. Genet. 22:421-477; Sanford et al. (1987) Particulate Science and Technology 5:27-37 (onion); Christou et al. (1988) Plant Physiol. 87:671-674 (soybean); McCabe et al. (1988) Biotechnology 6:923-926 (soybean); Datta et al. (1990) Biotechnology 8:736-740 (rice); Klein et al. (1988) Proc. Natl. Acad. Sci. USA 85:43054309 (maize); Klein et al. (1988) Biotechnology 6:559-563 (maize); WO 91/10725 (maize); Klein et al. (1988) Plant Physiol. 91:440444 (maize); Fromm et al. (1990) Biotechnology 8:833-839; and Gordon-Kamm et al. (1990) Plant Cell 2:603-618 (maize); Hooydaas-Van Slogteren & Hooykaas (1984) Nature (London) 311:763-764; Bytebier et al. (1987) Proc. Natl. Acad. Sci. USA 84:5345-5349 (Liliaceae); De Wet et al. (1985) In The Experimental Manipulation of Ovule Tissues, ed. G. P. Chapman et al., pp. 197-209. Longman, N.Y. (pollen); Kaeppler et al. (1990) Plant Cell Reports 9:415418; and Kaeppler et al. (1992) Theor. Appl. Genet. 84:560-566 (whisker-mediated transformation); U.S. Pat. No. 5,693,512 (sonication); D'Halluin et al. (1992) Plant Cell 4:1495-1505 (electroporation); Li et al. (1993) Plant Cell Reports 12:250-255 and Christou and Ford (1995) Annals of Botany 75:407413 (rice); Osjoda et al. (1996) Nature Biotechnology 14:745-750; Agrobacterium mediated maize transformation U.S. Pat. No. 5,981,840; silicon carbide whisker methods (Frame Br, Drayton P R, Bagnall S V, Lewnau J, Bullock W P, Wilson H M, Dunwell J M, Thompson J A and Wang K, 1994, Plant J. 6:941-948); laser methods (Guo Y, Liang, H and Berns M W, 1995, Physiologia Plantarum 93:19-24); sonication methods (Shiping Bao, Brian D. Thrall and Douglas L. Miller (1997) Ultrasound in Medicine & Biology 23:953-959; Finer K R, Finer J J (2000) Lett Appl Microbiol, 30:406-10; Amoah B K, Wu H, Sparks C, Jones H D, 2001 J Exp Bot 52:113542); polyethylene glycol mediated introduction of CONs in plant protoplasts (Krens R A, Molendijk L, Wullems G J and Schilperoort, RA, 1982, Nature 296:72-77); protoplasts of monocot and dicot cells can be transformed using electroporation (Fromm M, Taylor L P, Walbot V, 1985, Proc. Natl. Acad. Sci. USA 82:5824-5828) and microinjection (Crossway A, Oakes J V, Irvine J M, Ward B, Knauf V C and Shewmaker C K, 1986, Mol. Gen. Genet. 202, 179-185); all of which are herein incorporated by reference.

The target for transformation could be in the form of plant cells, tissues, or organs such as embryo, callus, leaf, inflorescence, root, shoot or seed. In other methods plant gametes, microspores, pollen, mother cells, zygote, or nucellar cells can be used. Such genomic modifications using oligonucleotides can also be performed in subcellular organelles such as chloroplasts and mitochondria.

Plants cells transformed with a plant expression vector can be regenerated, e.g., from single cells, callus tissue or leaf discs according to standard plant tissue culture techniques. Various cells, tissues, and organs from almost any plant can be successfully cultured to regenerate an entire plant. Plant regeneration from cultured protoplasts is described in Evans et al., Protoplasts Isolation and Culture, Handbook of Plant Cell Culture, Macmillan Publishing Company, New York, pp. 124-176 (1983); and Binding, Regeneration of Plants, Plant Protoplasts, CRC Press, Boca Raton, pp. 21-73 (1985).

The regeneration of plants containing the foreign gene introduced by Agrobacterium can be achieved as described by Horsch et al., Science 227:1229-1231 (1985) and Fraley et al., Proc. Natl. Acad. Sci. USA 80:4803 (1983). This procedure typically produces shoots within two to four weeks and these transformant shoots are then transferred to an appropriate root-inducing medium containing the selective agent and an antibiotic to prevent bacterial growth. Transgenic plants of the present invention may be fertile or sterile.

Regeneration can also be obtained from plant callus, explants, organs, or parts thereof. Such regeneration techniques are described generally in Klee et al., Ann. Rev. of Plant Phys. 38:467-486 (1987). The regeneration of plants from either single plant protoplasts or various explants is well known in the art. See, for example, Methods for Plant Molecular Biology, A. Weissbach and H. Weissbach, eds., Academic Press, Inc., San Diego, Calif. (1988). For maize cell culture and regeneration see generally, The Maize Handbook, Freeling and Walbot, Eds., Springer, N.Y. (1994); Corn and Corn Improvement, 3^{rd} edition, Sprague and Dudley Eds., American Society of Agronomy, Madison, Wis. (1988).

The cells, which have been altered by any targeted gene modification method, may also be grown into plants in accordance with conventional ways. See, for example, McCormick et al. (1986) Plant Cell Reports 5:81-84 and Gruber et. al., 1993, “Vectors for Plant Transformation” In: Methods in Plant Molecular Biology and Biotechnology; Glick and Thompson, eds., CRC Press, Inc., Boca Raton, pages 89-119; Gordon-Kamm et al., The Plant Cell 2:603-618 (1990). These plants may then be grown, and either pollinated with the same transformed strain or different strains, and the resulting hybrid having the desired phenotypic characteristic identified. Two or more generations may be grown to ensure that the subject phenotypic characteristic is stably maintained and inherited.

In one embodiment, a transgenic plant that is homozygous for the added heterologous nucleic acid is produced; i.e., a transgenic plant that contains two added nucleic acid sequences, one gene at the same locus on each chromosome of a chromosome pair. A homozygous transgenic plant can be obtained by sexually mating (selfing) a heterozygous transgenic plant that contains a single added heterologous nucleic acid, germinating some of the seed produced and analyzing the resulting plants produced for altered expression of a polynucleotide of the present invention relative to a control plant (i.e., native, non-transgenic). Backcrossing to a parental plant and out-crossing with a non-transgenic plant are also contemplated.

It may be necessary to transfect animal or lower eukaryotic cells. To do this, animal and lower eukaryotic (e.g., yeast) host cells are competent or rendered competent for transfection by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextrin, electroporation, biolistics, and micro-injection of the DNA directly into the cells. The transfected cells are cultured by means well known in the art. Kuchler, R. J., Biochemical Methods in Cell Culture and Virology, Dowden, Hutchinson and Ross, Inc (1997).

The present invention will be further described by reference to the following detailed examples. It is understood, however, that there are many extensions, variations, and modifications on the basic theme of the present invention beyond that shown in the examples and description, which are within the spirit and scope of the present invention.

### EXAMPLE 1

### Reducing the Expression of the Y1 Gene of Maize

Synthesis of Oligonucleotides Containing Modified Nucleic Analogs

Synthesis of oligonucleotides containing nucleic acid analogs, such as cationic or LNA oligonucleotides, can be performed using methods reported earlier (Dagle et al. (1990) Nucleic Acid Research 18:4751-4757; Dagle and Weeks (1996), Nucleic Acid Research 24:2143-2149; Weeks D L and Dagle J (1998); and U.S. Pat. No. 5,734,040). The actual sequence of a oligonucleotides, containing a nucleic acid analog, will depend on the sequence of the genomic target to be modified. The oligonucleotide sequence can be complementary to the “minus” or non-transcribed strand of the region of the gene to be modified or it can be complementary to the transcribed or “plus” strand of the region of the gene to be modified. The gene modification efficiency of the oligonucleotides may be different for the transcribed (or “plus”) DNA strand than that for the non-transcribed (or “minus”) DNA strand. In addition to the genomic target sequence to be modified, another factor, which affects the composition of cationic oligonucleotides, will be the number of cationic phosphoramidite internucleoside linkages in a specific cationic oligonucleotide molecule. Thus, a specific cationic oligonucleotide may contain one or more cationic phosphoramidite internucleoside linkages such than the total amount of cationic phosphoramidite internucleoside linkages in a specific cationic oligonucleotide may range up to 100%. Empirical determination of optimal conditions for specific applications of the cationic oligonucleotides can readily be determined. LNA-containing oligonucleotides can be analyzed by reversed phase-HPLC or capillary electrophoresis. Wahlestedt et. al., Proc. Natl. Acad. Sci. USA 97:5633-5638 (2000).

The phytoene synthase gene is one of the genes involved in the production of β-carotene in the endosperm and leaves of maize. Buckner et. al., The Plant Cell 2:867-876 (1990) and Buckneret. al., Genetics 143:479-488 (1996), both of which are hereby incorporated by reference. The sequence for the phytoene synthase gene can be found in SEQ ID NO: 1 and under GenBank/EMBL Data Bank with accession number U32636, which is herein incorporated by reference. The base within the phytoene synthase gene to be modified can be found at position number 1500 and is illustrated in SEQ ID NO: 2. The modifications are illustrated below:

Target Sequence From the Phytoene Synthase Gene:

| | |
|---|---|
| 5′ GGACTAAATAGATTCTAAAGTCATAAATACAT | (SEQ ID NO: 2) |
| TG [symbol] CCAAAGACTCAAATACCCTT 3′ | |

DNA residues in bold illustrate the RDS conserved domains. The DNA residue in bold and italics is the residue to be modified from a T to an A.

Oligonucleotide Sequence:

| | |
|---|---|
| 5′ TTAAATACATTG [symbol] CCAAAGACTCAATTTTTTTa | (SEQ ID NO: 3) |
| aattgagtctttggtcaatgtatttaaGCGCGTTTT | |
| CGCGC 3′ | |

Normal DNA residues are in upper case. The modified cationic or LNA residues are in lower case. The residue in bold and italics refers to the residue to be modified from T to A.

Introduction of Cationic or LNA Oligonucleotides into Plant Cells

An appropriate cationic or LNA oligonucleotide can be introduced into maize cells capable of growth on suitable maize culture medium. Such competent cells can be from maize suspension culture, callus culture on solid medium, freshly isolated immature embryos or meristem cells. Immature embryos of the Hi-II genotype can be used as the target cells. Ears are harvested at approximately 10 days post-pollination, and 1.2-1.5 mm immature embryos are isolated from the kernels, and placed scutellum-side down on maize culture medium.

The immature embryos are bombarded from 18-72 hours after being harvested from the ear. Between 6 and 18 hours prior to bombardment, the immature embryos are placed on medium with additional osmoticum (MS basal medium, Musashige and Skoog, 1962, Physiol. Plant 15:473497, with 0.25 M sorbitol). The embryos on the high-osmotic medium are used as the bombardment target, and are left on this medium for an additional 18 hours after bombardment.

For particle bombardment, DNA (described above) is precipitated onto 1.8 μm tungsten particles using standard CaCl₂— spermidine chemistry (see, for example, Klein et al., 1987, Nature 327:70-73). Each plate is bombarded once at 650 PSI, using a DuPont Helium Gun (Lowe et al., 1995, Bio/Technol 13:677-682). For typical media formulations used for maize immature embryo isolation, callus initiation, callus proliferation and regeneration of plants, see Armstrong, C., 1994, In “The Maize Handbook”, M. Freeling and V. Walbot, eds. Springer Verlag, N.Y., pp 663-671.

Within 1-7 days after particle bombardment, the embryos are moved onto N6-based culture medium containing no selection. Embryos, and later callus, are transferred to fresh media every 2 weeks. The calli developing from the immature embryos are screened.

Molecular Analysis of Genomic Modification of Phytoene Synthase Gene

In order to determine whether the above-described modification to the phytoene synthase gene has been accomplished, a PCR-based high-throughput assay will be performed to screen the transformants. Specifically, two oligonulcoetide primers (forward and reverse) will be designed based on the phytoene synthase cDNA sequence. Genomic DNA extracted from regenerating callus tissue from each transformation event will be used as template. The PCR reactions will be performed using the HotStarTaq Master Mix Kits for Qiagen, Inc. following the manufacturer's instructions. NucleoFast 96 PCR kits from BD BioSciences Clonetech, Inc. will be used for rapid and efficient purification of the PCR products. The purified products will be sequenced using ABI BigDye Terminator chemistry and the ABI 3700 Automatic DNA sequencer. One pair of PCR primers based on the phytoene synthase cDNA sequence is shown below.

| | |
|---|---|
| Forward Primer: | |
| 5′ GTTAAATGGGGCAACATCAG 3′ | (SEQ ID NO: 4) |
| Reverse Primer | |
| 5′ CAGAGTATATTCTAAGGG 3′ | (SEQ ID NO: 5) |

The expected 274 base pair PCR product will be purified and sequenced as described above. Presence of the base A in the PCR product corresponding to position number 1500 in the phytoene synthase cDNA sequence or presence of an ambiguous T/A base call at this position would indicate that the intended genomic modification has taken place.

The effect of this genomic modification on the stability of phytoene synthase mRNA will be confirmed by evaluation of the steady-state levels of phytoene synthase mRNA in the transformants using RT-PCR. Extraction and purification of total RNA from the transformants will be performed using the RNAEasy Plant Mini kits from Qiagen, Inc. The same set of primers as shown above (SEQ ID NOS: 5 and 6) will be used to carry out the RT-PCR reactions using the OneStep RT-PCR kits from Qiagen, Inc. following manufacturer's instructions. Maize actin will be used as an internal control to normalize the RNA levels from different samples.

The same set of primers as shown above (SEQ ID NOS: 5 and 6) could also be used in other high-throughput methods of quantitative RNA analysis such as Q-PCR. For review see, Ginzinger D G (2002) Exp Hematol 30:503-12 and references therein. Finally, Northern blot analysis (using standard protocols, such as described in Luehrsen, 1994, In “The Maize Handbook”, M. Freeling and V. Walbot, eds. Springer Verlag, N.Y., pp 572-574) will be performed to further confirm the decreased levels of phytoene synthase mRNA in the transformants.

Enzymatic assays for phytoene synthase will be performed using tissue extracts from transformants following the method described by Fraser et. al. (Fraser (2002) Proc. Natl. Acad. Sci. USA, 99:1092-97). Reduced levels of enzyme are also an indication of reduced expression or gene silencing.

### EXAMPLE 2

### Reducing the Expression of the Hebicide Safener-Binding Protien of Maize

Synthesis of Oligonucleotides Containing Modified Nucleic Analogs

Synthesis of oligonucleotides containing nucleic acid analogs, such as cationic or LNA oligonucleotides, can be performed using methods reported earlier (Dagle et al. (1990) Nucleic Acid Research 18:47514757; Dagle and Weeks (1996), Nucleic Acid Research 24:2143-2149; Weeks D L and Dagle J (1998); and U.S. Pat. No. 5,734,040). The actual sequence of a oligonucleotides, containing a nucleic acid analog, will depend on the sequence of the genomic target to be modified. The oligonucleotide sequence can be complementary to the “minus” or non-transcribed strand of the region of the gene to be modified or it can be complementary to the transcribed or “plus” strand of the region of the gene to be modified. The gene modification efficiency of the oligonucleotides may be different for the transcribed (or “plus”) DNA strand than that for the non-transcribed (or “minus”) DNA strand. In addition to the genomic target sequence to be modified, another factor, which affects the composition of cationic oligonucleotides, will be the number of cationic phosphoramidite internucleoside linkages in a specific cationic oligonucleotide molecule. Thus, a specific cationic oligonucleotide may contain one or more cationic phosphoramidite internucleoside linkages such than the total amount of cationic phosphoramidite internucleoside linkages in a specific cationic oligonucleotide may range up to 100%. Empirical determination of optimal conditions for specific applications of the cationic oligonucleotides can readily be determined. LNA-containing oligonucleotides can be analyzed by reversed phase-HPLC or capillary electrophoresis. Wahlestedt et. al., Proc. Natl. Acad. Sci. USA 97:5633-5638 (2000).

The maize herbicide safener-binding gene is involved in protecting maize against injury from chloroacetanilide and thiocarbamate herbicides. Please see, Scott-Craig et. al., Plant Physiol. 116:1083-1089 (1998), which is hereby incorporated by reference. The sequence for the maize herbicide safener-binding gene can be found in SEQ ID NO: 6 and under GenBank/EMBL Data Bank with accession number AF033496, which is herein incorporated by reference. The base within the gene to be modified can be found at position number 1138 and is illustrated in SEQ ID NO: 6. The modifications are illustrated below:

Target sequence of the Maize Herbicide Safener-Binding Gene:

| | |
|---|---|
| 5′ GGACTATACATATATATTTACAT ACATATA | (SEQ ID NO: 7) |
| TGTAT [symbol] TGTGTGGGTGCCTTGCGTGG 3′ | |

DNA residues in bold illustrate the RDS conserved domains. The DNA residue in bold and italics is the residue to be modified from a G to an C.

Oligonucleotide Sequence:

| | |
|---|---|
| 5′ ACATATATGTAT [symbol] TGTGTGGGTGCCTTTTTTTa | (SEQ ID NO: 8) |
| aaggcacccacacagatacatatatgtGCGCGTTTT | |
| CGCGC 3′ | |

Normal DNA residues are in upper case. The modified cationic or LNA residues are in lower case. The residue in bold and italics refers to the residue to be modified from G to C.

Introduction of Cationic or LNA Oligonucleotides into Plant Cells

An appropriate cationic or LNA oligonucleotide can be introduced into maize cells capable of growth on suitable maize culture medium. Such competent cells can be from maize suspension culture, callus culture on solid medium, freshly isolated immature embryos or meristem cells. Immature embryos of the Hi-II genotype can be used as the target cells. Ears are harvested at approximately 10 days post-pollination, and 1.2-1.5 mm immature embryos are isolated from the kernels, and placed scutellum-side down on maize culture medium.

The immature embryos are bombarded from 18-72 hours after being harvested from the ear. Between 6 and 18 hours prior to bombardment, the immature embryos are placed on medium with additional osmoticum (MS basal medium, Musashige and Skoog, 1962, Physiol. Plant 15:473497, with 0.25 M sorbitol). The embryos on the high-osmotic medium are used as the bombardment target, and are left on this medium for an additional 18 hours after bombardment.

For particle bombardment, DNA (described above) is precipitated onto 1.8 μm tungsten particles using standard CaCl₂— spermidine chemistry (see, for example, Klein et al., 1987, Nature 327:70-73). Each plate is bombarded once at 650 PSI, using a DuPont Helium Gun (Lowe et al., 1995, Bio/Technol 13:677-682). For typical media formulations used for maize immature embryo isolation, callus initiation, callus proliferation and regeneration of plants, see Armstrong, C., 1994, In “The Maize Handbook”, M. Freeling and V. Walbot, eds. Springer Verlag, N.Y., pp 663-671.

Within 1-7 days after particle bombardment, the embryos are moved onto N6-based culture medium containing no selection. Embryos, and later callus, are transferred to fresh media every 2 weeks. The calli developing from the immature embryos are screened.

Molecular Analysis of Genomic Modification of Maize Herbicide Safener-Binding Gene

In order to determine whether the above-described modification to the gene has been accomplished, a PCR-based high-throughput assay will be performed to screen the transformants. Specifically, two oligonulcoetide primers (forward and reverse) will be designed based on the herbicide safener-binding cDNA sequence. Genomic DNA extracted from regenerating callus tissue from each transformation event will be used as template. The PCR reactions will be performed using the HotStarTaq Master Mix Kits for Qiagen, Inc. following the manufacturer's instructions. NucleoFast 96 PCR kits from BD BioSciences Clonetech, Inc. will be used for rapid and efficient purification of the PCR products. The purified products will be sequenced using ABI BigDye Terminator chemistry and the ABI 3700 Automatic DNA sequencer. One pair of PCR primers based on the herbicide safener-binding cDNA sequence is shown below.

| | |
|---|---|
| Forward Primer: | |
| TTATGATTGACCACTCTGGGCC | (SEQ ID NO: 9) |
| Reverse Primer | |
| CCACGAGAGCCTTAAGAAAGCATC | (SEQ ID NO: 10) |

The expected 362 base pair PCR product will be purified and sequenced as described above. Presence of the base A in the PCR product corresponding to position number 1138 in the herbicide safener-binding cDNA sequence or presence of an ambiguous T/A base call at this position would indicate that the intended genomic modification has taken place.

The effect of the above-described genomic modification on the stability of herbicide safener-binding mRNA will be confirmed by evaluation of the steady-state levels of herbicide safener-binding mRNA in the transformants using RT-PCR. Extraction and purification of total RNA from the transformants will be performed using the RNAEasy Plant Mini kits from Qiagen, Inc. The same set of primers as shown above (SEQ ID NOS: 9 and 10) will be used to carry out the RT-PCR reactions using the OneStep RT-PCR kits from Qiagen, Inc. following manufacturer's instructions. Maize actin will be used as an internal control to normalize the RNA levels from different samples.

The same set of primers as shown above (SEQ ID NOS: 9 and 10) could also be used in other high-throughput methods of quantitative RNA analysis such as Q-PCR. For review see, Ginzinger D G (2002) Exp Hematol 30:503-12 and references therein. Finally, Northern blot analysis (using standard protocols, such as described in Luehrsen, 1994, In “The Maize Handbook”, M. Freeling and V. Walbot, eds. Springer Verlag, N.Y., pp 572-574) will be performed to further confirm the decreased levels of phytoene synthase mRNA in the transformants.

Analysis of safener binding protein levels will be performed using herbicide safener binding assays essentially as described by Scott-Craig et. al. in Scott-Craig et. al., Plant Physiol. 116:1083-9 (1998), which is hereby incorporated by reference.

All publications cited in this application are herein incorporated by reference to the same extent as if each individual publication or patent application was specifically and individually indicated to be incorporated by reference.

What is claimed is:

1. A method of reducing expression of a target polynucleotide in a plant cell, the method comprising, altering the target polynucleotide in the plant cell by targeted gene modification to produce an active RDS element within the targeted polynucleotide, wherein expression of the target polynucleotide is reduced.

2. The method of claim 1, wherein the plant cell is a monocot plant cell.

3. The method of claim 1, wherein the targeted gene modification is performed by a site-specific recombination method.

4. The method of claim 1, wherein the targeted gene modification is performed by a method using an oligonucleotide.

5. The method of claim 4, wherein the oligonucleotide contains a modified base.

6. The method of claim 4, wherein the oligonucleotide contains a cationic residue.

7. The method of claim 4, wherein the oligonucleotide contains a linked nucleic acid residue.

8. A plant cell made by the method of claim 1.

9. A method of destabilizing mRNA encoded by a target polynucleotide in a plant cell, the method comprising:
a. introducing an oligonucleotide into the plant cell, said oligonucleotide being designed to produce at least one active RDS element in the target polynucleotide;
b. maintaining the oligonucleotide within the nucleus of the plant cell whereby the mRNA of the target polynucleotide is destabilized.

10. The method of claim 9, wherein the oligonucleotide contains at least one modified base.

11. The method of claim 9, wherein the oligonucleotides contains a cationic residue.

12. The method of claim 9, wherein the oligonucleotides contains a linked nucleic acid residue.

13. The method of claim 19, wherein the oligonucleotide is selected from the group consisting of SEQ ID NO: 4 and SEQ ID NO: 8.

14. A plant cell made by the method of claim 9.

15. A method of reducing expression of a target polynucleotide in a plant cell, the method comprising:
a. introducing an oligonucleotide into the plant cell, said oligonucleotide being designed to produce an active RDS element within the target polynucleotide;
b. maintaining the oligonucleotide within the nucleus of the plant cell whereby the expression of the target polynucleotide is reduced.

16. The method of claim 15, wherein the oligonucleotide contains at least one modified base.

17. The method of claim 15, wherein the oligonucleotides contains a cationic residue.

18. The method of claim 15, wherein the oligonucleotides contains a linked nucleic acid residue.

19. The method of claim 15, wherein the oligonucleotide is selected from the group consisting of SEQ ID NO: 4 and SEQ ID NO: 8.

20. A plant cell made by the method of claim 15.
